(19) United States
(12) Patent Application Publication
(10) Pub. No.: US 2019/0126019 A1
(43) Pub. Date: May 2, 2019

(54) PELVIC TISSUE VISCOELASTIC STRETCHING DEVICE

(71) Applicant: EURL CORNIER, Paris (FR)

(72) Inventor: Edgard CORNIER, Paris (FR)

(21) Appl. No.: 16/142,333

(22) Filed: Sep. 26, 2018

(30) Foreign Application Priority Data
Oct. 31, 2017 (FR) 1760263

(51) Int. Cl. A61M 29/02

(52) U.S. Cl. CPC A61M 29/02; A61M 25/1002; A61M 2210/1475

(57) ABSTRACT

The pelvic tissue viscoelastic stretching device before childbirth consists of, on the one hand, a flexible basic body (1) in the form of an inverted quadrangular pyramidal structure extending in a support and positioning tab (4) and comprising an upper face (5) designed to be positioned beneath the uterus and a smaller lower face (6) designed to be positioned near the perineum, connected by an inactive front face (7) and by three active stretching faces, that is a posterior lower face (8) and two lateral faces (9) located on either side of it, and on the other, by three balloons (2, 3) respectively attached to the stretching faces (8, 9) of the basic body (1) and which can be inflated, in particular with physiological serum, to ensure posterior pressing against the sacrum and lateral pressing on the lifting muscles, up to an overall volume corresponding essentially to the head of a baby on birth.

### TECHNICAL FIELD

This invention concerns a device designed to ensure the viscoelastic stretching of pelvic tissue for its preparation prior to childbirth.

### BACKGROUND ART

During childbirth, the posterior bony part of the true pelvis consisting of the sacrum and the coccyx moves backwards (bone nutation) and the lateral and posterior lower tissues forming a viscoelastic whole are stretched downwards and rearwards.

The physical stretching model of this type of tissue indicates that it is elastic up to a certain limit then becomes anelastic, meaning that the tissue does not return to its initial shape and that there would be residual elongation.

If the stretching is excessive, the tissue limit, known as the complacency limit, is reached and causes tissue rupture and irreversible tearing which can have serious consequences; however, gradual and repeated anelastic stretching of biological tissue which repairs itself in position leads to enlargement without rupture.

Note that to avoid such tearing, it has already been proposed that before childbirth, midwives could carry out maneuvers to render the perineum flexible and increase its dilation by manual preventive methods, although this would not guarantee a result in that these maneuvers only concern the posterior lower part and not the lateral part of the perineum.

Various more or less “artisan” instrumental techniques ranging from African gourds to the “Epino” inflation system have also been used to the same end, but again without obtaining lateral and posterior lower stretching of the tissue in a differentiated manner depending on the biological location (lateral or posterior) that is gradual, controllable and adapted to the individual anatomy.

### DISCLOSURE OF THE INVENTION

In this context, the purpose of the present invention is to propose a pelvic tissue viscoelastic stretching device designed to avoid tissue rupture by pushing back the elastic stretching limit and the complacency limit of these tissues, prior to childbirth.

A device such as this, conforming to the invention, essentially consists of a flexible basic body and three balloon devices which can be inflated, in particular with physiological serum, to assume a position and an overall volume corresponding to that resulting from successive dilation sessions essentially to a baby's head on birth, having a diameter of approximately 9 cm.

According to the intervention, the basic body is essentially in the form of an inverted quadrangular pyramidal structure extending in a support and positioning tab comprising a wide upper face designed to be positioned beneath the uterus and a smaller lower face designed to be positioned near the perineum.

The upper face and the lower face of the basic body are connected by an inactive anterior face and by three active stretching faces, that is a is posterior face turned downward and to the rear and two lateral faces located on either side of it.

As far as the three balloons are concerned, they are attached to the stretching faces of the basic body and can be inflated to press, for the posterior balloon, against the sacrum and the posterior lower part of the elevating muscles, and for the lateral balloons, laterally against the lifting muscles.

These balloons can be attached to the stretching faces of the basic body or form an integral part of these faces.

The whole thus formed by the basic body and the three balloons must be made of a biocompatible material, such as silicon, and may be single-purpose or sterilizable.

According to a preferential characteristic of the invention, the upper face of the basic body is essentially rectangular with the larger sides corresponding to the lateral stretching faces while the lower face of this body is essentially square.

Note that the faces of the basic body are preferably concave.

Preferably, the height of the basic body is approximately 4.5 cm while the upper face is preferably an approximately 4 cm by 5 cm sided rectangle and that the lower face preferably comprises an approximately 3 cm-sided square.

The anterior, inactive face of the basic body is generally hollowed in the form of a cylinder section to protect the urinary urethra.

According to the invention, in the inflated condition, the balloon, attached to the posterior stretching face has a volume larger than that of the two balloons attached to the lateral stretching faces and is situated below these two lateral balloons.

Preferably, the posterior balloon is liable to be inflated to reach a diameter of approximately 7.5 cm without resistance whereas the two lateral balloons which are placed opposite one another can reach a diameter of approximately 4.5 cm without resistance.

These balloons can be inflated by inflating tubes supported by the support and positioning tab.

Advantageously, the device comprises a tube for the simultaneous inflation of the two lateral balloons and an independent inflation tube for the posterior balloon; however, the three balloons can be connected together.

After the insertion of the basic body into the vagina, the two lateral balloons are generally first inflated to allow the prior positioning of the device, then the posterior balloon is inflated in turn.

The three balloons can also be inflated simultaneously and each of them adapts to the individual morphology.

The lateral balloons inflate higher than the posterior lower balloon because of the rectangular shape of the upper base of the truncated pyramid supporting the structure, and the square shape which is smaller in the lower part of the support structure.

During this operation, the two lateral balloons and the weight of the uterus hold the posterior balloon and direct it downwards and to the rear, so that the sacrum-perineum structure can be lowered, while adjusting to the individual variations in the shape of the true pelvis.

The stretching pressure therefore applies consistently to the muscle mass of the lifting muscles.

As already indicated, the purpose according to the invention is to obtain, after several gradual stretching sessions to the inflated condition, an assembly comprising the basic body and the balloons, capable of reaching a 9-cm diameter without resistance, corresponding to the head of a baby on birth.

In accordance with a preferential characteristic of the invention, the stretching device also comprises managing devices for controlling, testing and regulating the inflation of the balloons, liable to be connected to the inflation tubes.

Preferably, these management devices are designed to allow the programming of the inflation volume of the balloons, and the speed and duration of inflation, according to the data recorded in a memory.

According to another characteristic of the invention, the support and positioning tab is concave towards the front.

This tab plays many functions consisting in particular in favoring the insertion of the device into the vagina and locating the inactive anterior face and the active posterior face of the basic body, to guarantee the correct positioning of the device.

The support and positioning tab is also designed to hold the device down, after the prior inflation of the lateral balloons and before the inflation of the posterior balloon, in particular by attachment to the stirrups of the examination table, or to facilitate the extraction of the device after the deflation of the balloons.

The device conforming to the invention produces preventive, gradual and controlled stretching of the pelvic tissue during a succession of sessions preparing for childbirth.

During each of these sessions, the balloons are inflated to reach a tolerable pressure that does not cause pain to the patient who may herself stop the process and the inflation parameters (volume, duration, time) are recorded in the memory of the managing devices: this is the elastic limit.

Then, additional uncomfortable inflation but that is not painful is generated, which will be held for a moment (for instance, 10% additional volume for two minutes), causing anelastic distension, leading on to slight enlargement.

The elongation of the tissue persists from one session to another but scarring occurs, causing the forming of new elastic tissues which increase the overall volume and that therefore permit greater enlargement and stretching of the tissues to be obtained during the following sessions.

The device conforming to the invention can thus be used, in steps and in several sessions, to bring the perineum to a new level of preventive stretching allowing a baby's head to pass without reaching the complacency threshold, and therefore without any irreversible tissue tearing.

Stretching is specifically gradual and differential in the thickest posterior lateral parts of the pelvis and in the thinner and fragile posterior lower part.

After the birth, the tissue recovers its elastic potential while preserving a part of the elongation caused; physiological involution then occurs without any sequels of rupture scarring.

The device conforming to the invention thus makes it possible to gradually reach the required dimensions without tissue tearing or muscle pull, causing irreversible scars; untorn but stretched tissue can recover its essential properties during the process of hormone decrease, or when accompanied by is therapeutic physical stimulations, or muscle exercise.

### BRIEF DESCRIPTION OF THE DRAWINGS

The characteristics of the device forming the subject of this invention are described in greater detail with reference to the attached non-limiting drawings in which:

FIG. 1 is a longitudinal schematic view of the stretching device,

FIG. 2 is a top view of this stretching device,

FIG. 3 is a lateral view of this stretching device.

### PREFERRED EMBODIMENT FOR CARRYING OUT THE INVENTION

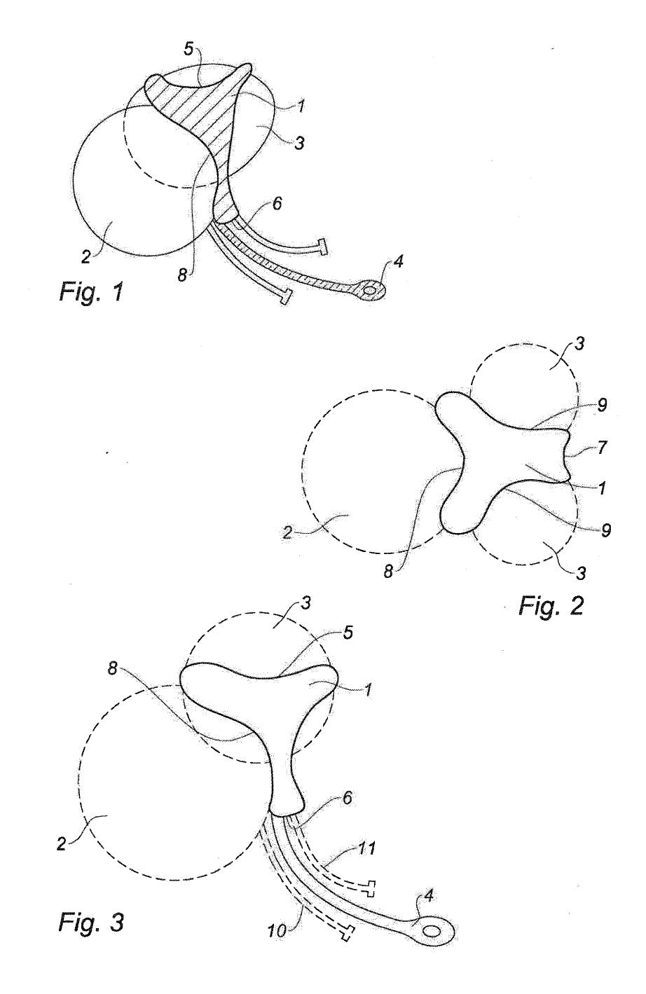

According to FIGS. 1, 2 and 3, the device comprises essentially a flexible basic body 1 and three balloons liable to be filled with physiological serum, that is, a posterior balloon 2 and two lateral balloons 3.

The basic body 1 is essentially in the form of an inverted quadrangular pyramidal section and extends in a support and positioning tab 4 which is concave towards the front.

The basic body 1 comprises more specifically a large upper face 5, essentially rectangular in shape, designed to be positioned under the uterus and a smaller lower face 6, essentially square, designed to be placed towards the perineum.

The upper face 5 and the lower face 6 of the basic body 1 are connected by an inactive anterior face 7 hollowed out in the form of a cylindrical section to protect the urinary urethra and by three active stretching faces, that is a posterior face 8 turned downward and to the rear and two lateral faces 9 located on either side of it.

The faces 5, 6, 7, 8 and 9 of the basic body 1 are concave.

The three balloons 2, 3 are respectively attached to the posterior face 8 and the lateral faces 9 of the basic body 1.

The support and positioning tab 4 supports a tube 10 for inflating the posterior balloon 2 and a tube 11 for the simultaneous inflation of the two lateral balloons 3.

In the inflated condition, the posterior balloon 2, attached to the posterior stretching face 8, has a volume larger than that of the two balloons 3 attached to the lateral stretching faces 9 and is situated below these two lateral balloons 3.

What is claimed is:

1. A device for pelvic tissue viscoelastic stretching prior to childbirth, characterized in that it comprises:
on the one hand, a flexible basic body (1), in the form of an inverted quadrangular pyramidal structure extending in a support and positioning tab (4) and comprising a wide upper face (5) designed to be positioned beneath the perineum, connected by an inactive anterior face (7) and by three active stretching faces, that is, a posterior face (8) turned toward the rear and the bottom and two lateral faces (9) on either side of it, and
on the other hand, by three balloons (2, 3) respectively attached to the stretching faces (8, 9) of the basic body and which can be inflated, in particular with physiological serum, to ensure posterior pressing against the sacrum and lateral pressure against the lifting muscles, to reach an overall volume corresponding essentially to the head of a baby on childbirth.

2. The device of claim 1,
characterized in that
the upper face (5) of the basic body (1) is essentially rectangular with the larger sides corresponding to the lateral stretching faces (9) while the lower face (6) of this body is essentially square.

3. The device of claim 1,
characterized in that
in the inflated condition, the balloon (2), attached to the posterior stretching face (8) has a volume larger than that of the two balloons (3) attached to the lateral stretching faces (9) and is situated below these two lateral balloons.

4. The device of claim 1,
characterized in that
it comprises a tube for the simultaneous inflation (11) of the two lateral balloons (3) and an independent inflation tube (10) for the posterior balloon (4), the three balloons being supported by the support and positioning tab (4).

5. The device of claim 4,
characterized in that
it comprises management devices allowing the control, testing and regulation of the inflation of the balloons (2, 3).

6. The device of claim 1,
characterized in that
the support and positioning tab (4) is concave toward the front.

7. The device of claim 1,
characterized in that
the faces of the basic body (1) are concave.

8. The device of claim 1,
characterized in that
the inactive anterior face (7) is hollowed in the form of a cylinder section to ensure the protection of the urinary urethra.

9. The device of claim 1,
characterized in that
it consists of sterilized double or single purpose biocompatible material.
